(19) United States
(12) Patent Application Publication
(10) Pub. No.: US 2003/0198983 A1
(43) Pub. Date: Oct. 23, 2003

(54) Methods of genetic analysis of human genes

(75) Inventor: Xue Mei Zhou, San Jose, CA (US)

Correspondence Address: PARSONS BEHLE & LATIMER, 201 S. MAIN STREET, SUITE 1800, SALT LAKE CITY, UT, 84111-2218, US

(73) Assignee: Affymetrix, Inc., Santa Clara, CA

(21) Appl. No.: 10/355,577

(22) Filed: Jan. 31, 2003

Related U.S. Application Data
(60) Provisional application No. 60/353,987, filed on Feb. 1, 2002.

(51) Int. Cl. C12Q 1/68; C12M 1/34

(52) U.S. Cl. 435/6; 435/287.2

(57) ABSTRACT

Nucleic acid sequences are disclosed which are complementary to a wide variety of Human genes. The sequences could be used for a variety of analyses. As such, methods of using the disclosed nucleic acid sequences are related to diverse fields impacted by the nature of molecular interaction, including chemistry, biology, medicine, and medical diagnostics.

### CLAIM OF PRIORITY

This application claims the benefit of U.S. Provisional Application No. 60/353,987, filed Feb.1, 2002.

### SEQUENCE LISTING

This application includes a sequence listing on compact disc and a computer readable form. The sequence listing information recorded in computer readable form is identical to the written (on compact disc) sequence listing.

### BACKGROUND

The following disclosure involves a unique pool of nucleic acid sequences useful for analyzing molecular interactions of biological interest. The subject matter therefore relates to diverse fields impacted by the nature of molecular interaction, including chemistry, biology, medicine, and medical diagnostics.

Many biological functions are carried out by regulating the expression levels of various genes, either through changes in levels of transcription (e.g. through control of initiation, provision of RNA precursors, RNA processing, etc.) of particular genes, through changes in the copy number of the genetic DNA, or through changes in protein synthesis. For example, control of the cell cycle and cell differentiation, as well as diseases, are characterized by the variations in the transcription levels of a group of genes.

Gene expression is not only responsible for physiological functions, but also associated with pathogenesis. For example, the lack of sufficient functional tumor suppressor genes and/or the over expression of oncogene/ protooncogenes leads to tumorgenesis. See, e.g., Marshall, Cell, 64: 313-326 (1991) and Weinberg, Science, 254: 1138-1146 (1991). Thus, changes in the expression levels of particular genes (e.g. oncogenes or tumor suppressors), serve as signposts for the presence and progression of various diseases. As a consequence, novel techniques and apparatus are needed to study gene expression in specific biological systems.

All documents, i.e., publications and patent applications, cited in this disclosure, including the foregoing, are incorporated by reference herein in their entireties for all purposes to the same extent as if each of the individual documents was specifically and individually indicated to be so incorporated by reference herein in its entirety.

### SUMMARY

Embodiments disclosed herein provide nucleic acid sequences that are complementary to particular Human genes and expressed sequence tags (ESTs) and apply them to a variety of analyses, including, for example, gene expression analysis. For example, one embodiment includes an array comprising any 10 or more, 100 or more, 1000 or more, 10,000 or more, 100,000 or more, or even more nucleic acid probes containing 9 or more consecutive nucleotides from the sequences listed in SEQ ID NOS: 1-997,516 or the perfect sense match, sense mismatch, perfect antisense match or antisense mismatch thereof. Another embodiment comprises the use of any of the above arrays, nucleic acid sequences or portions of the nucleic acid sequences disclosed in SEQ ID NOS: 1-997,516 to monitor gene expression levels by hybridization of the array to a DNA library; monitor gene expression levels by hybridization to an mRNA-protein fusion compound; identify polymorphisms; identify biallelic markers; produce genetic maps; analyze genetic variation; comparatively analyze gene expression, gene families or gene conservation between different species; analyze differential gene expression due to treatments including drug treatments, temperature shifts, or other alteration of other physiological parameters; analyze gene knockouts; or, to hybridize tag-labeled compounds. Still another embodiment is a method of analysis comprising hybridizing one or more pools of nucleic acids to two or more of the nucleic acid sequences or portions thereof disclosed in SEQ ID NOS: 1-997,156 and detecting said hybridization. Another embodiment comprises the use of any one or more of the nucleic acid sequences or portions thereof disclosed in SEQ ID NOS: 1-997,516 as a primer for polymerase chain reactions (PCR). Yet another embodiment comprises use of any one or more of the nucleic acids or portions thereof disclosed in SEQ ID NOS: 1-997,516 as a ligand.

### DETAILED DESCRIPTION

Definitions

Massive Parallel Screening: The phrase “massive parallel screening” refers to the simultaneous screening of at least 100, or greater than 1000, or greater than 10,000, or greater than 100,000, or more different nucleic acid hybridizations.

Nucleic Acid: The terms “nucleic acid” or “nucleic acid molecule” refer to a deoxyribonucleotide or ribonucleotide polymer in the conformations of nucleic acids including, but not limited to, either single-or double-stranded form. Unless otherwise limited, nucleic acids encompass all natural nucleotides or base analogs of natural nucleotides or bases. Nucleic acids include Peptide Nucleic Acids (PNAs). Nucleic acids are derived from a variety or sources including, but not limited to, naturally occurring nucleic acids, clones, or solution or solid phase synthesis.

Probe: As used herein a “probe” is defined as a nucleic acid capable of binding to a target nucleic acid of complementary sequence through one or more types of chemical bonds, usually through complementary base pairing.

As used herein, a probe may include natural bases (i.e. A, G, U, C, or T) or analog, modified or unusual bases whether synthetic or naturally occurring (7-deazaguanosine, inosine, etc.). In addition, the monomeric units in probes may be joined by a linkage other than a phosphodiester bond. Any portion of nucleic acids may be other than that found in nature. Thus, probes may be peptide nucleic acids in which the constituent bases are joined by peptide bonds rather than phosphodiester linkages. It is also envisioned that the definition of probes may include mixed nucleic acid peptide probes.

Target nucleic acid: The term “target nucleic acid” or “target sequence” refers to a nucleic acid or nucleic acid sequence that is to be analyzed. A target can be a nucleic acid to which a probe may hybridize. The probe may be specifically designed to hybridize to the target. It may be either the presence or absence of the target nucleic acid that is to be detected, or the amount of the target nucleic acid that is to be quantified. The term target nucleic acid may refer to the specific subsequence of a larger nucleic acid to which the probe is directed or to the overall sequence (e.g., gene or mRNA) whose expression level it is desired to detect. The difference in usage will be apparent from context.

mRNA or transcript: The term “mRNA” refers to transcripts of a gene. Transcripts are RNA including, for example, mature messenger RNA ready for translation, products of various stages of transcript processing. Transcript processing may include splicing, editing and degradation.

Subsequence: “Subsequence” refers to a sequence of nucleic acids that comprise a part of a longer sequence of nucleic acids.

Perfect match: The term “match,” “perfect match,” “perfect match probe” or “perfect match control” refers to a nucleic acid that has a sequence that is perfectly complementary to a particular target sequence. Sense and antisense sequences may be perfect matches to their respective complementary sequences. A sense sequence that is a perfect match may be referred to as a perfect sense match. An antisense sequence that is a perfect match may be referred to as a perfect antisense match. The nucleic acid is typically perfectly complementary to a portion (subsequence) of the target sequence. A perfect match (PM) probe can be a test probe, a normalization control probe, an expression level control probe, and the like. A perfect match control or perfect match is distinguished from a “mismatch” or “mismatch probe.”

Mismatch: The term “mismatch,” “mismatch control” or “mismatch probe” refers to a nucleic acid whose sequence is deliberately selected not to be perfectly complementary to a particular target sequence. As a non-limiting example, for each mismatch (MM) control in a high-density probe array there typically exists a corresponding perfect match (PM) probe that is perfectly complementary to the same particular target sequence. The mismatch may comprise one or more bases. Mismatch(es) may be located anywhere in the mismatch probe. In an embodiment, a single mismatch may be located at or near the center of the probe such that the mismatch is most likely to destabilize the duplex with the target sequence under the test hybridization conditions. A homo-mismatch substitutes an adenine (A) for a thymine (T) or vice versa or a guanine (G) for a cytosine (C) or vice versa. For example, if the target sequence was: 5′-AGGTCCA-3′, a probe designed with a single homo-mismatch at the central nucleotide would result in the following sequence: 3′-TCCTGGT-5′. A hetero-mismatch includes those mismatches that are not homo-mismatches.

If the central nucleotide, or the 5′ nucleotide of the central two nucleotides, of a sequence is defined as potential mismatch position zero, potential mismatch positions 3′ to position zero would be numbered as positive integers (1, 2, 3, etc.), while potential mismatch positions 5′ to position zero would be numbered as negative integers (−1, −2, −3, etc.). When a mismatch occurs at one of the potential mismatch positions, the numbered position may be referred to as mismatch position rather than a potential mismatch position. For example, in the above mismatch sequence, 3′TCCTGGT-5′, the mismatch occurs at mismatch position zero. A single mismatch is one where there is only one mismatched nucleotide within the sequence. A double mismatch is one where there are two mismatched nucleotides within the sequence. Similarly, there could be triple, quadruple or even higher levels of mismatch. A single sense mismatch is a sense sequence with one nucleotide mismatched. A single antisense mismatch is an antisense sequence with one nucleotide mismatched.

Array: An “array” is a solid support with at least a first surface having a plurality of different nucleic acid sequences attached to the first surface.

Gene Knockout: the term “gene knockout, ” as defined in Lodish et al., Molecular Cell Biology, (3d ed., Scientific American Books 1995), which is hereby incorporated by reference in its entirety for all purposes, is a technique for selectively inactivating a gene by replacing it with a mutant allele in an otherwise normal organism.

DNA Library: A DNA library may be a genomic library or a cDNA library. As used herein the term “genomic library” or “genomic DNA library” refers to a collection of cloned DNA molecules consisting of fragments of the entire genome (genomic library). DNA copies of the mRNA produced by a cell type may be a cDNA library, and a cDNA library may be inserted into a suitable cloning vector.

Polymorphism: “polymorphism” refers to the occurrence of two or more genetically determined alternative sequences or alleles in a population. A polymorphic marker or site is the locus at which divergence occurs. Some markers have at least two alleles, each occurring at a frequency of greater than one percent, and possibly greater than 10% or 20% of the selected population. A polymorphic locus may be as small as one base pair. Polymorphic markers include restriction fragment length polymorphisms, variable number or tandem repeats (VNTR's), hypervariable regions, minisatellites, dinucleotide repeats, trinucleotide repeats, tetranucleotide repeats, short tandem repeats, simple sequence repeats, and insertion elements such as ALU. The first identified allelic form is arbitrarily designated as the reference form and other allelic forms are designated as alternative or variant alleles. The allelic form occurring most frequently in a selected population is sometimes referred to as the wild type form. Diploid organisms may be homozygous or heterozygous for allelic forms. A diallelic or biallelic polymorphism has two forms. A triallelic polymorphism has three forms. A multiallelic polymorphism has N forms, where N could be any integer.

Genetic map: a “genetic map” is a map that presents the order of specific sequences on a chromosome or other genomic structures.

Genetic variation: “genetic variation” refers to variation in the sequence of the same region between two or more organisms.

Hybridization: the association of two complementary nucleic acid strands, nucleic acid and a nucleic acid derivative, or nucleic acid derivatives (such as peptide nucleic acid) to form double stranded molecules. Hybrids can contain two DNA strands, two RNA strands, or one DNA and one RNA strand. Additionally, hybrids can contain derivatives in any combination.

mRNA-protein fusion: a compound whereby an mRNA is directly attached to the peptide or protein it encodes by a stable covalent linkage.

Ligand: any molecule, that binds tightly and specifically to a macromolecule, for example, a protein, forming a macromolecule-ligand complex.

II. General

SEQ ID NOS: 1-997,516 are encompassed in the Sequence Listing. Each sequence from SEQ ID NOS: 1-997,516 corresponds to and represents at least four nucleic acid sequences included as part of this disclosure. For example, if the first nucleic acid sequence listed in SEQ ID NOS: 1-997,516 is 5′-cgtgc-3′ the sequences included in this disclosure which are represented by this nucleic acid sequence are, for example:

gcacg=perfect sense match;

gctcg=sense mismatch at the central position;

cgtgc=perfect antisense match; and

cgagc=antisense mismatch at the central position.

Accordingly, for each nucleic acid sequence listed in SEQ ID NOS: 1-997,516, this disclosure includes the corresponding perfect sense match, sense mismatch, perfect antisense match and antisense mismatch. The position of the mismatch is not limited to the above example, it may be located from mismatch position −5 to mismatch position 5 relative to the central position of the probe or position zero.

Consequently, the present disclosure includes: a) the sequences listed in SEQ ID NOS: 1-997,516, or the perfect sense match, sense mismatch, perfect antisense match or antisense mismatch thereof; b) clones which comprise the nucleic acid sequences listed in SEQ ID NOS: 1-997,516, or the perfect sense match, sense mismatch, perfect antisense match or antisense mismatch thereof; c) longer nucleotide sequences which include the nucleic acid sequences listed in SEQ ID NOS: 1-997,516, or the perfect sense match, sense mismatch, perfect antisense match or antisense mismatch thereof; and d) subsequences greater than 9 nucleotides in length of the nucleic acid sequences listed in SEQ ID NOS: 1-997,516, or the perfect sense match, sense mismatch, perfect antisense match or antisense mismatch thereof.

The sequences of SEQ ID NOS: 1-997,516 are deposited on NetAffx, which is accessible on the world wide web from http://www.Affvmetrix.com.

The present disclosure describes a pool of unique nucleotide sequences complementary to Human sequences in particular embodiments which alone, or in combinations of two or more, 10 or more, 100 or more, 1,000 or more, 10,000 or more, 100,000 or more, or even more, can be used for a variety of applications.

In an embodiment, this disclosure describes a pool of unique nucleotide sequences that are complementary to many human gene sequences suitable for array based massive parallel screening of gene expression.

Array based methods for monitoring gene expression are disclosed and discussed in detail in U.S. Pat. Nos. 5,800,992, 6,013,449, 6,020,135, 6,033,860, 6,040,138, 6,177,248 and 6,309,822 and PCT Publication No. WO 92/10588 (published on Jun. 25, 1992), each of which is incorporated herein by reference for all purposes. Generally those methods of monitoring gene expression involve (1) providing a pool of target nucleic acids comprising RNA transcript(s) of one or more target gene(s), or nucleic acids derived from the RNA transcript(s); (2) hybridizing the nucleic acid sample to a high density array of probes; and (3) detecting the hybridized nucleic acids and determining expression levels.

The development of Very Large Scale Immobilized Polymer Synthesis, or VLSIPS, technology has provided methods for making very large arrays of nucleic acid probes in very small arrays. See U.S. Pat. Nos. 5,143,854, 5,242,974, 5,252,743, 5,324,633, 5,384,261, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, and 6,136,269, in PCT Publication Nos. WO 90/15070 and 92/10092, in PCT Applications Nos. PCT/US99/00730 (International Publication Number WO 99/36760) and PCT/US 01/04285, in U.S. patent applications Ser. Nos. 09/501,099 and 09/122,216, and Fodor et al., Science, 251: 767-77 (1991), each of which is incorporated herein by reference. In addition, U.S. Pat. No. 5,800,992 describes methods for making arrays of nucleic acid probes that can be used to detect the presence of a nucleic acid containing a specific nucleotide sequence. Methods of forming high density arrays of nucleic acids, peptides and other polymer sequences with a minimal number of synthetic steps are known. The nucleic acid array can be synthesized on a solid substrate by a variety of methods, including, but not limited to, light-directed chemical coupling, and mechanically directed coupling.

In an embodiment of a detection method, an array of immobilized nucleic acids, or probes, is contacted with a sample containing target nucleic acids, where the target nucleic acids have a fluorescent label attached. Target nucleic acids hybridize to the probes on the array and any non-hybridized nucleic acids are removed. The array containing the hybridized target nucleic acids are exposed to light that excites the fluorescent label. The resulting fluorescent intensity, or brightness, is detected. Relative brightness is used to determine 1) which probe is the best candidate for the perfect match to the hybridized target, and 2) the relative concentration of those targets. Once the intensity of the perfect match probe is known, concentrations of the target relative to other experiments, or relative to other targets on the same array can be estimated.

In an embodiment an array of the probes are presented in pairs, one probe in each pair being a perfect match to the target sequence and the other probe being identical to the perfect match probe except that the central base, mismatch position zero, is a homo-mismatch. Mismatch probes provide a control for non-specific binding or cross-hybridization to a nucleic acid in the sample other than the target to which the probe is directed. Thus, mismatch probes indicate whether a hybridization is or is not specific. For example, if the target is present, the perfect match probes should be consistently brighter than the mismatch probes because fluorescence intensity, or brightness, corresponds to binding affinity. See, e.g., U.S. Pat. No. 5,324,633, which is incorporated by reference herein for all purposes. In addition, if all possible mismatches are present at a particular position, the mismatch probes could be used to detect a mutation. Finally the difference in intensity (I) between the perfect match (PM) and the mismatch (MM) probe (I(PM)-I(MM)) provides a good measure of the concentration of the hybridized material.

In an embodiment a pool of sequences is provided that may be used as probes for their complementary genes listed in the Unigene, GenBank or TIGR databases. Methods for making probes are well known. See, e.g., Sambrook, Fritsche and Maniatis, “Molecular Cloning: A laboratory Manual” (2d ed., Cold Spring Harbor Press 1989) (Maniatis et al.), which is hereby incorporated in its entirety by reference for all purposes. Maniatis et al. describes a number of uses for nucleic acid probes of defined sequence. Some of the uses described by Maniatis et al. include screening cDNA or genomic DNA libraries, or subclones derived from them, for additional clones containing segments of DNA that have been isolated and previously sequenced; in Southern, northern, or dot-blot hybridization, identifying or detecting the sequences of specific genes; in Southern, or dot-blot hybridization of genomic DNA, detecting specific mutations in genes of known sequence; detecting specific mutations generated by site-directed mutagenesis of cloned genes; and mapping the 5′ termini of mRNA molecules by primer extensions. Maniatis et al. describes other uses for probes throughout. See also, Alberts et al., Molecular Biology of the Cell, 307 (3d ed., Garland Publishing Inc. 1994) and Lodish et al., Molecular Cell Biology, 285-286 (3d ed., Scientific American Books 1995) (brief discussion of the use of nucleic acid probes in in situ hybridization), each of which is hereby incorporated by reference in its entirety for all purposes. Other uses for probes derived from the sequences disclosed herein will be readily apparent to those of skill in the art. See, e.g., Lodish et al., Molecular Cell Biology, 229-233 (3d ed., Scientific American Books 1995) (description of the construction of genomic libraries), incorporated above.

Embodiments disclosed herein may be combined with known methods to monitor expression levels of genes in a wide variety of contexts. For example, where the effects of a drug on gene expression are to be determined, the drug is administered to an organism, a tissue sample, or a cell and the gene expression levels are analyzed. For example, nucleic acids are isolated from treated and untreated tissue samples, cells, or biological samples from organisms. Those nucleic acids are hybridized to a high density probe array containing probes directed to the gene(s) of interest, corresponding gene expression levels are determined, and hybridization patterns between treated and untreated sources compared. The types of drugs that may be used in these types of experiments include, but are not limited to, antibiotics, antivirals, narcotics, anti-cancer drugs, tumor suppressing drugs, and any chemical composition that may affect the expression of genes in vivo or in vitro. Embodiments such as this are particularly suited for the types of analyses described by, for example, U.S. Pat. No. 6,309,822, which is incorporated by reference in its entirety for all purposes. Further, because mRNA hybridization correlates to gene expression level, hybridization patterns can be compared to determine differential gene expression. See Wodicka et al., Nature Biotechnology, 15 (1997), hereby incorporated by reference in its entirety for all purposes. As non-limiting examples: hybridization patterns from samples treated with certain types of drugs may be compared to hybridization patterns from samples that have not been treated or that have been treated with a different drug; hybridization patterns for samples infected with a specific virus may be compared against hybridization patterns from non-infected samples; hybridization patterns for samples with cancer may be compared against hybridization patterns for samples without cancer; hybridization patterns of samples from cancerous cells that have been treated with a tumor suppressing drug may be compared against untreated cancerous cells, etc. Zhang et al., Science, 276: 1268-1272, hereby incorporated by reference in its entirety for all purposes, provides an example of how gene expression data can provide a great deal of insight into cancer research. One skilled in the art will appreciate that a wide range of applications will be available using two or more, 10 or more, 100 or more, 1000 or more, 10,000 or more, 100,000 or more, or even more of the SEQ ID NOS: 1-997,516 sequences as probes for gene expression analysis. The combination of the DNA array technology and the Human specific probes in this disclosure is a powerful tool for studying gene expression.

In an embodiment, SEQ ID NOS: 1-997,516 may be used in conjunction with techniques that link specific proteins to the mRNA that encodes the specific protein. See, e.g., Roberts and Szostak, Proc. Natl. Acad. Sci., 94: 12297-12302 (1997), which is incorporated herein by reference in its entirety for all purposes. Hybridization of these mRNA-protein fusion compounds to arrays comprised of two or more, 10 or more, 100 or more, 1000 or more, 10,000 or more, 100,000 or more, or even more of the sequences disclosed herein provides a powerful tool for monitoring expression levels.

In an embodiment, a pool of unique nucleic acid sequences can be used for parallel analysis of gene expression under selective conditions. By way of illustration and in no way limiting, genetic analysis under selective conditions includes variation in the temperature of the organism's environment; variation in pH levels in the organism's environment; variation in an organism's food (type, texture, amount etc.); variation in an organism's surroundings; etc. Arrays, such as those in the present disclosure, can be used to determine whether gene expression is altered when an organism is exposed to selective conditions. The variation and parallel analysis could occur for one individual organism or to different samples of different populations or individuals of the organism.

### Methods for Using Nucleic Acid Arrays to Analyze Genetic Selections Under Selective Conditions

Cho et al., in Proc. Natl. Acad. Sci., 95: 3752-3757 1998), incorporated herein by reference in its entirety for all purposes, describes the use of a high-density array containing oligonucleotides complementary to every gene in the yeast Saccharomyces cerevisiae to perform protein-protein interaction screens for S. cerevisiae genes implicated in mRNA splicing and microtubule assembly. Cho et al. was able to characterize the results of a screen in a single experiment by hybridization of labeled DNA derived from positive clones. Briefly, as described by Cho et al., two proteins are expressed in yeast as fusions to either the DNA-binding domain or the activation domain of a transcription factor. Physical interaction of the two proteins reconstitutes transcriptional activity, turning on a gene essential for survival under selective conditions. In screening for novel protein-protein interactions, yeast cells are first transformed with a plasmid encoding a specific DNA-binding fusion protein. A plasmid library of activation domain fusions derived from genomic DNA is then introduced into these cells. Transcriptional activation fusions found in cells that survive selective conditions are considered to encode peptide domains that may interact with the DNA-binding domain fusion protein. Clones are then isolated from the two-hybrid screen and mixed into a single pool. Plasmid DNA is purified from the pooled clones and the gene inserts are amplified using PCR. The DNA products are then hybridized to yeast whole genome arrays for characterization. The methods employed by Cho et al. are applicable to the analysis of a range of genetic selections. High density arrays created using two or more, 10 or more, 100 or more, 1000 or more, 10,000 or more, 100,000 or more, or even more of the sequences disclosed herein can be used to analyze genetic selections in the Human system using the methods described in Cho et al.

In an embodiment, a pool of unique nucleic acid sequences that can be used to identify biallelic markers (and multiallelic markers other than biallelic, such as triallelic, as well) is disclosed, providing a novel and efficient approach to the study of genetic variation. For example, methods for using high density arrays comprised of probes which are complementary to the genomic DNA of a particular species to interrogate polymorphisms are well known. See, e.g., U.S. Pat. No. 6,300,063 and U.S. patent application Ser. No. 08/965,620, which are hereby incorporated by reference herein for all purposes. Pools of two or more, 10 or more, 100 or more, 1000 or more, 10,000 or more, 100,000 or more, or even more of the sequences disclosed herein combined with the methods described in the above patent applications provide tools for studying genetic variation in the Human system.

In an embodiment, genetic variation can be used to produce genetic maps. Winzeler et al., Direct Allelic Variation Scanning of the Yeast Genome, Science 5380: 1194-97 (Aug. 21, 1998), describes methods for conducting this type of screening with arrays containing probes complementary to the yeast genome, and is hereby incorporated herein by reference for all purposes. Briefly, genomic DNA from strains which are phenotypically different is isolated, fragmented, and labeled. Each strain is then hybridized to identical arrays comprised of the nucleic acid sequences complementary to the system being studied. Comparison of hybridization patterns between the various strains then serve as genetic markers. As described by Winzler et al, these markers can then be used for linkage analysis. High density arrays created from two or more, 10 or more, 100 or more, 1000 or more, 10,000 or more, 100,000 or more, or even more of the sequences disclosed herein can be used to study genetic variation using the methods described by Winzler et al.

In an embodiment, cross-species comparisons may be done. One skilled in the art will appreciate that it is often useful to determine whether a gene present in one species, for example Human, is present in a conserved format in another species, including, without limitation, mouse, chicken, zebrafish, Drosophila, Escherichia coli or yeast. See, e.g., Andersson et al., Mamm Genome, 7(10):717-734 (1996) (describing the utility of cross-species comparisons), which is hereby incorporated by reference for all purposes. The use of two or more, 10 or more, 100 or more, 1000 or more, 10,000 or more, 100,000 or more, or even more of the sequences disclosed herein in an array can be used to determine whether any of the sequence from one or more of Human genes represented by the sequences disclosed herein is conserved in another species by, for example, hybridizing genomic nucleic acid samples from another species to an array comprised of the sequences disclosed herein. Areas of hybridization will yield genomic regions where the nucleotide sequence is highly conserved between the interrogation species and the Human genome.

In an embodiment, the genotype of gene knockouts may be determined. Methods for using gene knockouts to identify a gene are well known. See, e.g., Lodish et al., Molecular Cell Biology, 292-96 (3d ed., Scientific American Books 1995) and U.S. Pat. No. 5,679,523, which are hereby incorporated by reference for all purposes. By isolating genomic nucleic acid samples from knockout species with a known phenotype and hybridizing the samples to an array comprised of two or more, 10 or more, 100 or more, 1000 or more, 10,000 or more, 100,000 or more, or even more of the sequences disclosed herein, candidate genes which contribute to the phenotype will be identified and made accessible for further characterization.

In an embodiment, new gene family members may be identified. Methods of screening libraries with probes are well known. See, e.g., Maniatis et al., incorporated by reference above. Because the disclosed sequences comprise nucleic acid sequences from specific known genes, two or more, 10 or more, 100 or more, 1000 or more, 10,000 or more, 100,000 or more, or even more of sequences disclosed herein may be used as probes to screen genomic libraries to look for additional family members of those genes from which the disclosed sequences are derived.

In an embodiment, the disclosed sequences may be used to provide nucleic acid sequences to be used as tag sequences. Tag sequences are a type of genetic “bar code” which can be used to label compounds of interest. The analysis of deletion mutants using tag sequences is described in, for example, Shoemaker et al., Nature Genetics, 14: 450-456 (1996), which is hereby incorporated by reference in its entirety for all purposes. Shoemaker et al. describes the use of PCR to generate large numbers of deletion strains. Each deletion strain is labeled with a unique 20-base tag sequence that can be hybridized to a high-density oligonucleotide array. The tags serve as unique identifiers (molecular bar codes) that allow analysis of large numbers of deletion strains simultaneously through selective growth conditions. The use of tag sequences need not be limited to this example. The utility of using unique known short oligonucleotide sequences capable of hybridizing to a nucleic acid array to label various compounds will be apparent to one skilled in the art. One or more, 10 or more, 100 or more, 1000 or more, 10,000 or more, 100,000 or more, or even more of the SEQ ID NOS: 1-997,516 sequences are excellent candidates to be used as tag sequences.

In an embodiment, the sequences disclosed herein may be used to generate primers directed to their corresponding genes or genomic sequences as disclosed in the GenBank or any other public database. These primers may be used in such basic techniques as sequencing or PCR. See, e.g., Maniatis et al., incorporated herein by reference above.

In an embodiment, the nucleic acid sequences disclosed herein can be used as ligands for specific genes. The sequences disclosed herein may be used as ligands to their corresponding genes as disclosed in the Genbank or any other public database. Compounds that specifically bind known genes are of interest for a variety of uses. One particular clinical use is to act as an antisense nucleic acid that specifically binds and disables a gene, or expression of that gene, which has been, for example, linked to a disease. Methods and uses for ligands to specific genes are known. See for example, U.S. Pat. No. 5,723,594, which is hereby incorporated by reference in its entirety for all purposes.

In an embodiment, the hybridized nucleic acids are detected by detecting one or more labels attached to the sample nucleic acids. The labels may be incorporated by any of a number of means well known to those of skill in the art. In an embodiment, the label is simultaneously incorporated during the amplification step in the preparation of the sample nucleic acids. For example, PCR with labeled primers or labeled nucleotides will provide a labeled amplification product. In an embodiment, transcription amplification, as described above, using a labeled nucleotide (e.g. fluorescein-labeled UTP and/or CTP) incorporates a label into the transcribed nucleic acids.

Alternatively, a label may be added directly to the original nucleic acid sample (e.g., mRNA, polyA mRNA, cDNA, etc.) or to the amplification product after the amplification is completed. Means of attaching labels to nucleic acids are well known to those of skill in the art and include, for example nick translation or end-labeling (e.g. with a labeled RNA) by kinasing of the nucleic acid and subsequent attachment (e.g. ligation) of a nucleic acid linker joining the sample nucleic acid to a label (e.g. a fluorophore).

Detectable labels suitable for use may include any composition detectable by spectroscopic, photochemical, biochemical, immunochemical, electrical, calorimetric, or physical means. Useful labels in the present disclosure include biotin for staining with labeled streptavidin conjugate, magnetic beads (e.g., DYNABEADS), fluorescent dyes (e.g., fluorescein, texas red, rhodamine, green fluorescent protein, and the like), radiolabels (e.g., ³H, ¹²⁵I, ³⁵S, ¹⁴C, or ³²P), phosphorescent labels, enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241, each of which is hereby incorporated by reference in its entirety for all purposes.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing (through naked eye visual inspection or the use of enhanced means) the colored label.

The label may be added to the target nucleic acid(s) prior to, or after the hybridization. “Direct labels” are detectable labels that are directly attached to or incorporated into the target nucleic acid prior to hybridization. In contrast, “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an aviden-conjugated fluorophore will bind the biotin bearing hybrid duplexes providing a label that is easily detected. For a detailed review of methods of labeling nucleic acids and detecting labeled hybridized nucleic acids see, P. Tijssen, Laboratory Techniques in Biochemistry and Molecular Biology, Vol. 24: Hybridization With Nucleic Acid Probes (ed. Elsevier, N.Y., 1993), which is hereby incorporated herein by reference in its entirety for all purposes.

Fluorescent labels are easily added during an in vitro transcription (IVT) reaction. In an embodiment, fluorescein labeled UTP and CTP are incorporated into the RNA produced in an IVT reaction as described above.

### EXAMPLE

The following example serves to illustrate a method of using the disclosed sequences, and does not limit any inventions described by the appended claims.

### Gene Expression Monitoring by Hybridization to High Density Oligonucleotide Arrays

Arrays containing the desired number of probes are synthesized using the method described in U.S. Pat. No. 5,143,854, incorporated by reference above. Extracted poly (A)⁺RNA is converted to cDNA using the methods described below. The cDNA is then transcribed in the presence of labeled ribonucleotide triphosphates. The label may include biotin or a dye such as fluorescein. RNA is then fragmented with heat in the presence of magnesium ions. Hybridizations are carried out in a flow cell that contains the two-dimensional DNA probe arrays. Following a brief washing step to remove unhybridized RNA, the arrays are scanned using a scanning confocal microscope.

1. A method of RNA preparation:

Labeled RNA is prepared from clones containing a T7 RNA polymerase promoter site by incorporating labeled ribonucleotides in an in vitro transcription (IVT) reaction as described in the GeneChip Expression Analysis Technical Manual, Affymetrix, Inc. 2003. Either biotin-labeled or fluorescein-labeled UTP and CTP (1:3 labeled to unlabeled) plus unlabeled ATP and GTP is used for the reaction with 2500 U of T7 RNA polymerase.

Following the reaction unincorporated nucleotide triphosphates are removed using size-selective membrane such as MICROCON - 100, (Amicon, Beverly, Mass.). The total molar concentration of RNA is based on a measurement of the absorbance at 260 nm, as known to one skilled in the art. Following quantitation of RNA amounts, RNA is fragmented randomly to an average length of approximately 50 bases by heating at 94° C. in 40 mM Tris-acetate pH 8.1, 100 mM potassium acetate, 30 mM magnesium acetate, for 30 to 40 minutes. Fragmentation reduces possible interference from RNA secondary structure, and minimizes the effects of multiple interactions with closely spaced probe molecules.

For material made directly from cellular RNA, cytoplasmic RNA is extracted from cells by the method of Favaloro et al., Methods Enzymol, 65:718-749 (1980), hereby incorporated by reference for all purposes, and poly (A)⁺RNA is isolated with an oligo dT selection step using, for example, POLY ATRACT, (Promega, Madison, Wis.). RNA can be amplified using a modification of the procedure described by Eberwine et al., Proc. Natl. Acad. Sci., USA 89:3010-3014 (1992), hereby incorporated by reference for all purposes. Microgram amounts of poly (A)⁺RNA are converted into double stranded cDNA using a cDNA synthesis kit (kits may be obtained from Life Technologies, Gaithersburg, Md.) with an oligo dT primer incorporating a T7 RNA polymerase promoter site. After second-strand synthesis, the reaction mixture is extracted with phenol/chloroform, and the double-stranded DNA isolated using a membrane filtration step using, for example, MICROCON -100, (Amicon). Labeled cRNA (RNA made from cDNA) can be made directly from the cDNA pool with an IVT step as described above. The total molar concentration of labeled cRNA is determined from the absorbance at 260 nm and assuming an average RNA size of 1000 ribonucleotides. As known to one skilled in the art, the commonly used convention is that 1 OD is equivalent to 40 μg of RNA, and that 1 μg of cellular mRNA consists of 3 pmol of RNA molecules. Cellular mRNA may also be labeled directly without any intermediate cDNA synthesis steps. In this case, Poly (A)⁺RNA is fragmented as described, and the 5′ ends of the fragments are kinased and then incubated overnight with a biotinylated oligoribonucleotide (5′-biotin-AAMAA-3′) in the presence of T4 RNA ligase (available from Epicentre Technologies, Madison, Wis.). Alternatively, mRNA has been labeled directly by UV-induced cross-linking to a psoralen derivative linked to biotin (available from Schleicher & Schuell, Keene, N.H.).

2. Array hybridization and Scanning:

Array hybridization solutions can be made containing 0.9 M NaCI, 60 mM EDTA, and 0.005% TRITON X-100, adjusted to pH 7.6 (referred to as 6×SSPE-T). In addition, the solutions should contain 0.5 mg/ml unlabeled, degraded herring sperm DNA (available from Sigma, St. Louis, Mo.). Prior to hybridization, RNA samples are heated in the hybridization solution to 99° C. for 10 minutes, placed on ice for 5 minutes, and allowed to equilibrate at room temperature before being placed in the hybridization flow cell. Following hybridization, the solutions are removed, the arrays washed with 6×SSPE-T at 22° C. for 7 minutes, and then washed with 0.5×SSPE-T at 40° C. for 15 minutes. When biotin labeled RNA is used the hybridized RNA should be stained with a streptavidin-phycoerythrin in 6×SSPE-T at 40° C. for 5 minutes. The arrays are read using a scanning confocal microscope made by Molecular Dynamics (commercially available through Affymetrix, Santa Clara, Calif.). The scanner uses an argon ion laser as the excitation source, with the emission detected by a photomultiplier tube through either a 530 nm bandpass filter (suitable to detect flourescein emission) or a 560 nm longpass filter (suitable to detect phycoerythrin emission). Nucleic acids of either sense or antisense orientations may be used in hybridization experiments. Arrays for probes with either orientation (reverse complements of each other) are made using the same set of photolithographic masks by reversing the order of the photochemical steps and incorporating the complementary nucleotide.

3. Quantitative analysis of hybridization patterns and intensities.

Following a quantitative scan of an array, a grid is aligned to the image using the known dimensions of the array and the corner control regions as markers. The image is then reduced to a simple text file containing position and intensity information using software developed at Affymetrix (available with the confocal scanner). This information is merged with another text file that contains information relating physical position on the array to probe sequence and the identity of the RNA (and the specific part of the RNA) for which the oligonucleotide probe is designed. The quantitative analysis of the hybridization results involves a simple form of pattern recognition based on the assumption that, in the presence of a specific RNA, the perfect match (PM) probes will hybridize more strongly on average than their mismatch (MM) partners. The number of instances in which the PM hybridization is larger than the MM signal is computed along with the average of the logarithm of the PM/MM ratios for each probe set. These values are used to make a decision (using a predefined decision matrix) concerning the presence or absence of an RNA. To determine the quantitative RNA abundance, the average of the difference (I(PM)-I(MM)) for each probe family is calculated. The advantage of the difference method is that signals from random cross-hybridization contribute equally, on average, to the PM and MM probes, while specific hybridization contributes more to the PM probes. By averaging the pairwise differences, the real signals add constructively while the contributions from cross-hybridization tend to cancel. When assessing the differences between two different RNA samples, the hybridization signals from side-by-side experiments on identically synthesized arrays are compared directly. The magnitude of the changes in the average of the difference (I(PM)-I(MM)) values is interpreted by comparison with the results of spiking experiments as well as the signals observed for the internal standard bacterial and phage RNAs spiked into each sample at a known amount. Data analysis programs, such as those described in U.S. patent application Ser. No. 08/828,952, (Publication No. 0183933 A1) perform these operations automatically.

### CONCLUSION

This disclosure includes a pool of unique nucleic acid sequences that are complementary to many human gene sequences. These sequences can be used for a variety of types of analyses.

The above description is illustrative and not restrictive. Many variations of the inventions will become apparent to those of skill in the art upon review of this disclosure. The scope of the inventions should, therefore, be determined not with reference to the above description, but instead with reference to the appended claims along with their full scope of equivalents.

I claim:

1. An array comprising a plurality of nucleic acid probes, wherein said plurality of nucleic acid probes comprises each of the sequences listed in SEQ ID NOS: 1-997,516 or a perfect sense match, a perfect antisense-match, a sense mismatch where a single mismatch occurs at a central position, or an antisense mismatch where a single mismatch occurs at a central position.

2. The array of claim 1 wherein said array is used to monitor gene expression levels by hybridization to a DNA library.

3. The array of claim 1 wherein said array is used for analysis of genetic variation.

4. The array of claim 1 wherein said array is used for hybridization of tag-labeled compounds.

5. The array of claim 1 wherein said nucleic acid probes are specifically designed for analysis of at least one target sequence.

6. The array of claim 1 wherein said plurality of nucleic acid probes is attached to a solid support.

7. A method of analysis comprising: hybridizing one or more nucleic acids to the array of claim 1 and detecting a hybridization pattern.

8. The method of claim 7 wherein said method of analysis comprises monitoring gene expression levels.

9. The method of claim 8 wherein said monitoring gene expression levels comprises comparing gene expression levels of nucleic acids derived from two or more different samples and further comprises the step of comparing said hybridization patterns between said nucleic acids derived from said two or more different samples.

10. The method of claim 7 wherein said method of analysis comprises identifying biallelic markers.

11. The method of claim 7 wherein said method of analysis comprises identifying polymorphisms.

12. The method of claim 7 wherein said method of analysis comprises a cross-species comparison wherein the hybridization patterns of a pool of nucleic acids derived from one species are compared with the hybridization patterns of a pool of nucleic acids derived from a another species.

13. The method of claim 7 wherein each of said nucleic acids further comprises a tag sequence.

14. The method of claim 7 wherein said method of analysis is a method of identifying family members of a gene.

15. A method comprising using a plurality of probes to probe a sample wherein the plurality of probes comprises each of the sequences listed in SEQ ID NOS: 1-997,516 or a perfect sense match, a perfect antisense match, a sense mismatch where a single mismatch occurs at a central position, or an antisense mismatch where a single mismatch occurs at a central position.

16. The method of claim 15 wherein said plurality of probes is used in an in situ hybridization.

17. The method of claim 15 wherein said plurality of probes is used to screen cDNA or genomic libraries, or subclones derived from cDNA or genomic libraries, for additional clones containing segments of DNA that have been isolated and previously sequenced.

18. The method of claim 15 wherein said plurality of probes is used in Southern, northern, or dot-blot hybridization to identify or detect the sequence of any gene.

19. The method of claim 15 wherein said plurality of probes is used in Southern or dot-blot hybridization of genomic DNA to detect specific mutations in any gene.

20. The method of claim 15 wherein said plurality of probes is used to map the 5′ termini of mRNA molecules by primer extensions.
